(19) United States
(12) Patent Application Publication
(10) Pub. No.: US 2012/0184438 A1
(43) Pub. Date: Jul. 19, 2012

(54) FORMULATIONS

(75) Inventors: Gordon Alistair Bell, Bracknell (GB); David Stock, Bracknell (GB); Jeffrey Steven Wailes, Bracknell (GB)

(73) Assignee: SYNGENTA CROP PROTECTION, LLC, Greensboro, NC (US)

(21) Appl. No.: 13/386,813

(22) PCT Filed: Jul. 15, 2010
(86) PCT No.: PCT/GB10/01353

§ 371 Date: Apr. 5, 2012

(30) Foreign Application Priority Data
Jul. 24, 2009 (GB) 0912975.0

(51) Int. Cl. A01N 43/46; A01N 43/40; A01N 43/36; A01N 39/02; A01N 37/10; C07D 223/04; C07D 265/30; C07D 211/60; C07D 207/16; C07C 233/65; A01P 13/00; A01P 7/04; A01P 21/00; A01P 3/00; A01N 43/84

(52) U.S. Cl. 504/220; 504/226; 504/249; 504/287; 504/317; 504/318; 540/607; 544/176; 546/245; 548/539; 564/175; 564/183

(57) ABSTRACT

This invention relates to agrochemical compositions comprising a benzamide compound of formula (I) where R¹ is fluoro, methoxy or C₁₋₄ alkyl; R² and R³ are each independently optionally substituted C₁₋₆ alkyl or optionally substituted C₂₋₆ alkenyl; or R³ is hydrogen and R³ is optionally substituted C₅₋₈ alkyl or optionally substituted C₄₋₆ alkenyl or optionally substituted benzyl; or R² and R³ and the nitrogen atom to which they are both attached together form an optionally substituted 5-, 6- or 7-membered ring, which ring optionally contains at least one further heteroatom selected from O, N and S; to the use of those benzamide compounds as solvents; to certain novel compounds of formula (I); and to a process for preparing those novel compounds.

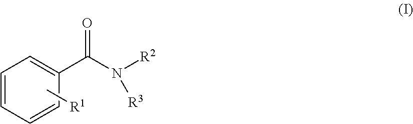

This invention relates to agrochemical compositions comprising certain benzamide compounds, to the use of those benzamide compounds as solvents, especially in formulations, particularly in agrochemical formulations and in environmentally friendly formulations; to certain novel benzamide compounds; and to a process to prepare those novel compounds.

Nowadays, the Formulation Chemist is required to address a number of criteria when developing new formulations. Ideally, a suitable solvent will display an excellent dissolving power for pesticides or other organic molecules. The compounds of the present invention display an excellent dissolving power; the compounds may be used effectively as solvents.

Accordingly the present invention provides a composition comprising an agrochemical and a compound of formula (I)

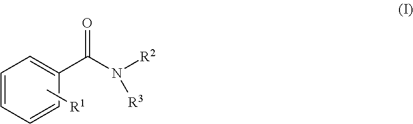

where R¹ is fluoro, methoxy or C₁₋₄ alkyl; R² and R³ are each independently optionally substituted C₁₋₆ alkyl or optionally substituted C₂₋₆ alkenyl; or R² is hydrogen and R³ is optionally substituted C₅₋₈ alkyl or optionally substituted C₄₋₆ alkenyl or optionally substituted benzyl; or R² and R³ and the nitrogen atom to which they are both attached together form an optionally substituted 5-, 6- or 7-membered ring, which ring optionally contains at least one further heteroatom selected from O, N and S.

In another aspect, the present invention provides the use of a compound of formula (I) as defined above as a solvent.

Alkyl groups and moieties are straight or branched chains. Examples are methyl, ethyl, iso-propyl, n-propyl, n-butyl, sec-butyl, tert-butyl, n-amyl and iso-amyl [3-methylbutyl].

Alkenyl groups and moieties may be in the form of straight or branched chains and, where appropriate, may be of either the (E)- or (Z)-configuration. Examples are vinyl and allyl.

Any optional substituents are independently selected.

Each optionally substituted alkyl group is an alkyl group optionally substituted by one or two phenyl groups, preferably one phenyl group.

Each alkyl group is suitably not substituted.

Each optionally substituted alkenyl group is an alkenyl group optionally substituted by one or two phenyl groups, preferably one phenyl group.

Each alkenyl group is suitably not substituted.

Optional substituents on each 5-, 6- or 7-membered ring are selected from C₁₋₄ alkyl; suitably selected from C₁₋₂ alkyl; and more suitably methyl.

When R³ is optionally substituted benzyl, suitable optional substituents on benzyl are one or more independently selected C₁₋₃ alkyl groups in the ortho, meta, para or methylene positions; and more suitably the optional substituents are each methyl.

Suitably R¹ is in the ortho- or meta-position; more suitably it is in the ortho-position.

Suitably R¹ is fluoro, methoxy or C₁₋₂ alkyl; more suitably R¹ is C₁₋₂ alkyl; even more suitably R¹ is methyl.

Suitably R² and R³ together comprise one to four carbon atoms.

In the situation where R² and R³ are each independently optionally substituted C₁₋₆ alkyl or optionally substituted C₂₋₆ alkenyl, then suitably R² and R³ are each independently C₁₋₆ alkyl or C₂₋₆ alkenyl, more suitably R² and R³ are each independently C₁₋₅ alkyl or C₃₋₄ alkenyl.

In the situation where R² is hydrogen and R³ is optionally substituted C₅₋₈ alkyl or optionally substituted C₄₋₆ alkenyl or optionally substituted benzyl, then suitably R³ is C₅₋₈ alkyl or C₄₋₆ alkenyl or benzyl; more suitably R³ is n-pentyl, 2-ethylhexyl or benzyl.

In the situation where R² and R³ and the nitrogen atom to which they are both attached together from an optionally substituted 5-, 6- or 7-membered ring, which ring optionally contains at least one further heteroatom selected from O, N and S, then suitably the at least one further heteroatom is selected from O and S; more suitably the at least one further heteroatom is O; and even more suitably the ring is morpholinyl.

Suitably the invention provides the use of a compound of formula (I) in an agrochemical formulation. Each compound of the present invention has a low water solubility and yet is a good solvent for agrochemicals.

Suitably the composition is a formulation concentrate which may be diluted or dispersed [typically in water] by an end-user [typically a farmer] in a spray tank prior to application.

Suitably the agrochemical in the composition of the present invention is a herbicide, fungicide, insecticide or plant growth regulator.

WO2009/130281A1 provides both agrochemicals and conventional formulation components which may be used in conjunction with the present invention; the teaching of WO2009/130281A1 is hereby incorporated by reference.

In particular, WO2009/130281A1 discloses from page 28, line 37 to page 45, line 32 agrochemical active ingredients suitable for use with the present invention. Of course, the various editions of The Pesticide Manual [especially the 14^{th} and 15^{th} editions] also disclose details of agrochemicals, any one of which may suitably be used with the present invention.

In particular, WO2009/130281A1 discloses from page 46, line 5 to page 51, line 40 standard formulation components suitable for use with the present invention [despite the fact that WO2009/130281A1 relates to adjuvants whereas the present invention relates to solvents]. Of course other standard formulation publications also disclose formulation components suitable for use with the present invention [for example, Chemistry and Technology of Agrochemical Formulations, Ed. Alan Knowles, published by Kluwer Academic Publishers, The Netherlands in 1998; and Adjuvants and Additives: 2006 Edition by Alan Knowles, Agrow Report DS256, published by Informa UK Ltd, December 2006].

Suitable herbicides include bicyclopyrone, mesotrione, fomesafen, tralkoxydim, napropamide, amitraz, propanil, pyrimethanil, dicloran, tecnazene, toclofos methyl, flamprop M, 2,4-D, MCPA, mecoprop, clodinafop-propargyl, cyhalofop-butyl, diclofop methyl, haloxyfop, quizalofop-P, indol-3-ylacetic acid, 1-naphthylacetic acid, isoxaben, tebutam, chlorthal dimethyl, benomyl, benfuresate, dicamba, dichlobenil, benazolin, triazoxide, fluazuron, teflubenzuron, phenmedipham, acetochlor, alachlor, metolachlor, pretilachlor, thenylchlor, alloxydim, butroxydim, clethodim, cyclodim, sethoxydim, tepraloxydim, pendimethalin, dinoterb, bifenox, oxyfluorfen, acifluorfen, fluoroglycofen-ethyl, bromoxynil, ioxynil, imazamethabenz-methyl, imazapyr, imazaquin, imazethapyr, imazapic, imazamox, flumioxazin, flumiclorac-pentyl, picloram, amodosulfuron, chlorsulfuron, nicosulfuron, rimsulfuron, triasulfuron, triallate, pebulate, prosulfocarb, molinate, atrazine, simazine, cyanazine, ametryn, prometryn, terbuthylazine, terbutryn, sulcotrione, isoproturon, linuron, fenuron, chlorotoluron and metoxuron.

Suitable fungicides include isopyrazam, mandipropamid, azoxystrobin, trifloxystrobin, kresoxim methyl, famoxadone, metominostrobin and picoxystrobin, cyprodanil, carbendazim, thiabendazole, dimethomorph, vinclozolin, iprodione, dithiocarbamate, imazalil, prochloraz, fluquinconazole, epoxiconazole, flutriafol, azaconazole, bitertanol, bromuconazole, cyproconazole, difenoconazole, hexaconazole, paclobutrazole, propiconazole, tebuconazole, triadimefon, trtiticonazole, fenpropimorph, tridemorph, fenpropidin, mancozeb, metiram, chlorothalonil, thiram, ziram, captafol, captan, folpet, fluazinam, flutolanil, carboxin, metalaxyl, bupirimate, ethirimol, dimoxystrobin, fluoxastrobin, orysastrobin, metominostrobin and prothioconazole.

Suitable insecticides include thiamethoxam, imidacloprid, acetamiprid, clothianidin, dinotefuran, nitenpyram, fipronil, abamectin, emamectin, bendiocarb, carbaryl, fenoxycarb, isoprocarb, pirimicarb, propoxur, xylylcarb, asulam, chlorpropham, endosulfan, heptachlor, tebufenozide, bensultap, diethofencarb, pirimiphos methyl, aldicarb, methomyl, cyprmethrin, bioallethrin, deltamethrin, lambda cyhalothrin, cyhalothrin, cyfluthrin, fenvalerate, imiprothrin, permethrin and halfenprox.

Suitable plant growth regulators include paclobutrazole and 1-methylcyclopropene. The composition of the present invention may also comprise one or more surfactants or dispersing agents to assist the emulsification of the agrochemical on dispersion or dilution in an aqueous medium (dispersant system). The emulsification system is present primarily to assist in maintaining the emulsified agrochemical in water. Many individual emulsifiers, surfactants and mixtures thereof suitable for forming an emulsion system for an agrochemical are known to those skilled in the art and a very wide range of choices is available. Typical surfactants that may be used to form an emulsifier system include those containing ethylene oxide, propylene oxide or ethylene oxide and propylene oxide; aryl or alkylaryl sulphonates and combinations of these with either ethylene oxide or propylene oxide or both; carboxylates and combinations of these with either ethylene oxide or propylene oxide or both. Polymers and copolymers are also commonly used.

Compositions of the present invention may also include other solvents, which may have a range of water solubilitites. Oils with very low water solubilities may be added to the solvent of the present invention for assorted reasons such as the provision of scent, biological enhancement, safening, cost reduction, improvement of the emulsification properties and alteration of the solubilising power. Solvents with higher water solubility may also be added for various reasons, for instance to alter the ease with which the formulation emulsifies in water, to improve the solubility of the pesticide or of the other optional additives in the formulation, to change the viscosity of the formulation or to add a commercial benefit.

Other optional ingredients which may be added to the formulation include colourants, scents, safeners, and other materials which benefit a typical agrochemical formulation.

Agrochemical emulsion concentrate formulations (ECs) are solutions of a pesticide which emulsify when added to agricultural spray tanks full of water. The selection of suitable solvents is complicated by the need to have good solvent power while also having a low enough aqueous solubility to prevent dissolution in the spray tank. Water insoluble oils tend to be poorer solvents for agrochemicals than water soluble solvents. An oil that is also a good solvent is therefore very valuable for these formulations. The invention provides a series of compounds which have a solubility in water that is low, typically less than 0.1% w/w in water, and which are also able to dissolve high concentrations of pesticides.

In addition to ECs, other suitable formulation types include EWs [oil-in-water emulsions], SEs [suspoemulsions], CSs [capsule suspensions] and SLs [soluble concentrates].

Suitably the compound of formula (I) has an aqueous solubility at 25° C. which is less than 5% w/w; more suitably less than 2.5% w/w; even more suitably less than 0.5% w/w; and most suitably less than 0.1% w/w.

Suitably the solubility of the agrochemical in the compound of formula (I) at 25° C. is greater than 5% w/w; more suitably greater than 10% w/w; and even more suitably greater than 20% w/w.

Many of the compounds disclosed by the present invention are novel.

Therefore in a further aspect, the present invention provides a compound of formula (I) as defined above provided (i) that when R¹ is methyl in the meta-position, then R² is not ethyl when R³ is ethyl; and (ii) that when R¹ is methyl in the ortho-position, then R³ is not n-pentyl nor benzyl when R² is hydrogen; R³ is not n-propyl when R² is n-propyl; R² is not methyl when R³ is tert-butyl; R³ is not methyl when R² is tert-butyl; and R² and R³ and the nitrogen atom to which they are both attached do not together form pyrrolidinyl, morpholinyl or 2,6-dimethylmorpholinyl.

The compounds of the present invention may be prepared by reacting a suitable amine with a suitable acid chloride, acid anhydride or ester.

Therefore in another aspect, the present invention provides a process for preparing a compound of formula (I) as defined above comprising the step of reacting a compound of formula (II) with a compound of formula (III)

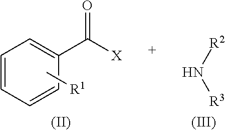

where X is Cl, OY or

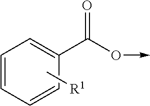

and Y is a leaving group, such as methoxy, ethoxy, propoxy (linear or branched), butoxy (linear or branched) or paratoluene sulphonyl; and R¹, R² and R³ are as defined above.

Table 1 provides structures of suitable solvents and shows selected ¹H NMR (400 MHz) data, all obtained with CDCl₃ as the solvent. The following abbreviations are used throughout this description:

“NMR” = nuclear magnetic resonance spectrum.

| | | | |
|---|---|---|---|
| s = | singlet | br = | broad |
| d = | doublet | dd = | doublet of doublets |
| t = | triplet | q = | quartet |
| m = | multiplet | ppm = | parts per million |

TABLE 1

| Com- | | ¹H-NMR data: |
|---|---|---|
| pound | | (ppm/number of |
| No. | Structure | Hs/multiplicity) |
| 1 | 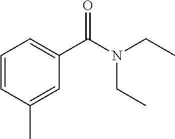 | N/A |
| 2 | 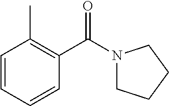 | 7.30-7.15 (4H, m); 3.65 (2H, t); 3.15 (2H, t); 2.30 (3H, s); 2.00 (2H, m); 1.90 (2H, m). |
| 3 | 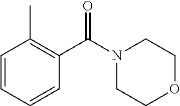 | 7.30-7.15 (4H, m); 3.85 (2H, m); 3.80 (2H, m); 3.60 (2H, m); 3.25 (2H, m); 2.30 (3H, s). |
| 4 | 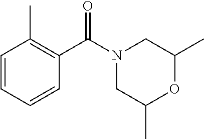 | 7.30-7.15 (4H, m); 4.65 (1H, br); 3.65 (1H, br); 3.50 (1H, br); 3.25 (1H, br); 2.75 (1H, br); 2.55 (1H, dd); 2.35 and 2.30 (3H, 2br. s); 1.30 (3H, d); 1.05 (3H, d). |
| 5 | 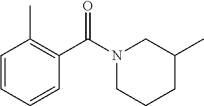 | (very complex due to restricted rotation) 7.30-7.10 (4H, m); 4.60, 3.35, 2.80 & 2.75- 2.40 (4H, 4m); 2.30 (3H, d and s); 1.90-1.10 (5H, m); 1.00 and 0.75 (3H, d and m). |
| 6 | 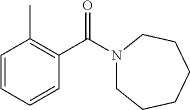 | 7.30-7.10 (4H, m); 3.90- 3.50 (2H, br); 3.25 (2H, br); 2.30 (3H, s); 1.95-1.80 (2H, br); 1.70-1.50 (6H, br). |
| 7 | 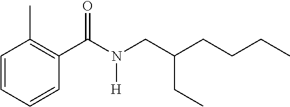 | 7.35-7.15 (4H, m); 5.70 (1H, br); 3.40 (2H, m); 2.45 (3H, s); 1.55 (1H, m); 1.40 (2H, m); 1.35-1.25 (6H, m); 1.00-0.90 (6H, m). |
| 8 | 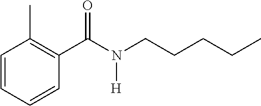 | 7.35-7.25 (2H, m); 7.20- 7.15 (2H, m); 5.80 (1H, br); 3.40 (2H, m); 2.45 (3H, s); 1.60 (2H, m); 1.35 (4H, m); 0.95 (3H, t). |
| 9 | 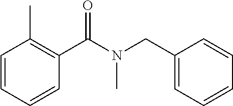 | (complex due to restricted rotation) 7.40-7.10 (9H, m), 4.80 and 4.35 (2H, 2br), 3.05 and 2.50 (3H, 2s), 2.30 (3H, 2s). |
| 10 | 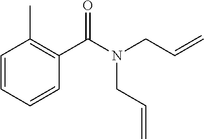 | 7.30-7.15 (4H, m); 5.90 (1H, m); 5.65 (1H, m); 5.30-5.05 (4H, m); 4.50-3.80 (2H, br); 3.70 (2H, d); 2.30 (3H, s). |
| 11 | 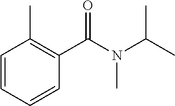 | (complex due to restricted rotation) 7.30-7.10 (4H, m); 5.05 & 3.75 (1H, 2m); 3.00 & 2.65 (3H, 2s); 2.30 (3H, 2s); 1.15 & 1.10 (6H, d and m). |
| 12 | 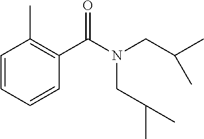 | 7.30-7.15 (4H, m); 3.60- 3.20 (2H, br); 3.10-2.85 (2H, br); 2.30 (3H, s); 2.15 (1H, m); 1.85 (1H, m) 1.00 (6H, d); 0.75 (6H, d). |
| 13 | 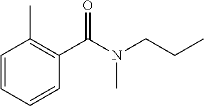 | (complex due to restricted rotation) 7.30-7.10 (4H, m); 3.55 and 3.05 (2H, br and t); 3.10 and 2.80 (3H, 2s); 2.30 (3H, 2s); 1.70 and 1.50 (2H, 2m); 1.00 and 0.75 (3H, 2t). |
| 14 | 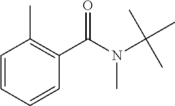 | 7.25-7.10 (4H, m); 2.75 (3H, s); 2.30 (3H, s); 1.55 (9H, s). |
| 15 | 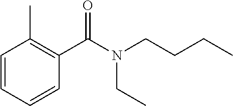 | (complex due to restricted rotation) 7.30-7.15 (4H, m); 3.80-3.20 (2H, br); 3.15 and 3.05 (2H, 2m); 2.30 (3H, 2s); 1.70, 1.45 and 1.10 (4H, 3m); 1.25, 1.05, 1.00 and 0.75 (6H, 4t). |
| 16 | 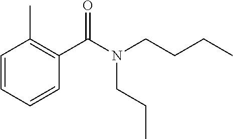 | (complex due to restricted rotation) 7.30-7.10 (4H, m); 3.80-3.10 (2H, br); 3.05 (2H, m); 2.30 (3H, s); 1.70, 1.45 and 1.10 (6H, 3m); 1.00 (3H, m); 0.75 (3H, m). |
| 17 | 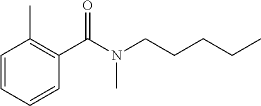 | (complex due to restricted rotation) 7.30-7.15 (4H, m); 3.55 and 3.10 (2H, br and t); 3.10 and 2.80 (3H, 2s); 2.30 (3H, s); 1.70, 1.50, 1.40-1.35, 1.20 and 1.10 (6H, 5m); 0.95 and 0.80 (3H, 2t). |
| 18 | 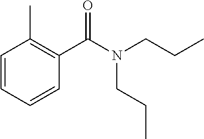 | 7.30-7.15 (4H, m); 3.80- 3.20 (2H, br); 3.00 (2H, br); 2.30 (3H, s); 1.70 (2H, m); 1.50 (2H, m); 1.00 (3H, t); 0.75 (3H, t). |
| 19 | 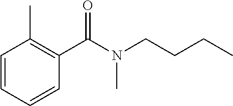 | (complex due to restricted rotation) 7.30-7.10 (4H, m); 3.55 and 3.10 (2H, br and t); 3.10 and 2.80 (3H, 2s); 2.30 (3H, s); 1.65, 1.50- 1.40 and 1.15 (4H, 3m); 1.00 and 0.80 (3H, 2t). |
| 20 | 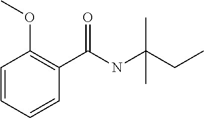 | ¹H NMR (400 MHz, CDCl₃) 8.10 (2H, dd), 7.70 (1H, br), 7.30 (1H, dt), 6.95 (1H, dt), 6.85 (1H, d), 3.85 (3H, s), 1.75 (2H, q), 1.35 (6H, s), 0.85 (3H, t). |
| 21 | 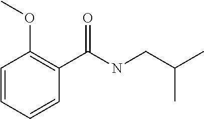 | ¹H NMR (400 MHz, CDCl₃) 8.05 (1H, dd), 7.85 (1H, br), 7.25 (1H, dt), 6.90 (1H, t), 6.80 (1H, d), 3.80 (3H, s), 3.15 (2H, t), 1.80 (1H, m), 0.85 (6H, m). |
| 22 | 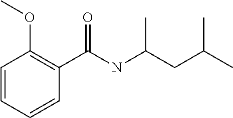 | ¹H NMR (400 MHz, CDCl₃) 8.10 (1H, dd), 7.60 (1H, br d), 7.35 (1H, dt), 6.95 (1H, t), 6.85 (1H, d), 4.20 (1H, m), 3.85 (3H, s), 1.75 (1H, m), 1.40 (1H, m), 1.30 (1H, m), 1.15 (3H, d), 0.90 (6H, d). |
| 23 | 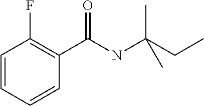 | ¹H NMR (400 MHz, CDCl₃) 7.95 (1H, dt), 7.35 (1H, m), 7.15 (1H, t), 7.00 (1H, dd), 6.45 (1H, br), 1.75 (2H, q), 1.35 (6H, s), 0.85 (3H, t). |
| 24 | 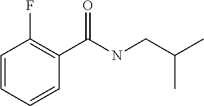 | ¹H NMR (400 MHz, CDCl₃) 7.85 (1H, dt), 7.30 (1H, m), 7.10 (1H, t), 6.95 (1H, dd), 6.90 (1H, br), 3.20 (2H, dt), 1.80 (1H, m), 0.85 (6H, s). |
| 25 | 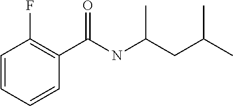 | ¹H NMR (400 MHz, CDCl₃) 7.95 (1H, dt), 7.35 (1H, m), 7.15 (1H, t), 7.00 (1H, dd), 6.55 (1H, br), 4.25 (1H, m), 1.65 (1H, m), 1.45 (1H, m), 1.30 (1H, m), 1.20 (3H, d), 0.90 (6H, m). |

The compounds of the invention may be used in a variety of end use applications (including agrochemical formulations), particularly as solvents. These solvents may be used with a wide variety of materials, including herbicides, fungicides, acaricides, nematicides and insecticides [and also plant growth regulators].

The compounds of the invention may be used to formulate solutions of a variety of materials, including agrochemicals, which may be formulated as emulsion or dispersion concentrates, emulsions in water or oil, microencapsulated formulations, aerosol sprays or fogging formulations; and these may be further formulated into granular materials or powders, for example for dry application or as water-dispersible formulations. The solutions so formed may also be used directly on soil or plants or in other non-agrochemical applications.

Examples of such applications include paper making, water treatment, forestry applications, public health treatments, use in municipal pools and other water courses, in applications near rivers, lakes, reservoirs or seas and in applications where release to the atmosphere has to be minimised or controlled and where damage to the atmosphere is not desirable. Examples include use in exterior and interior paints, coatings, varnishes, waxes or other protectant layers or opacifiers, colourants or screens; in dyeing, pigmentation or the use of inks; in cleaning products designed for the home, garden or industrial applications; and in soap or detergent applications for industrial, home or environmental usage. The compounds of the present invention may also be used in shampoos, household detergency and in household cleaners [for example oven cleaners and surface cleaners].

The compounds of the present invention have exceptional dissolving power for a wide variety of agrochemicals, pharmaceuticals and other commercially valuable compounds, plus the dissolving power also extends to dissolution of dirt, grease or waxes.

The invention is illustrated by the following Examples in which:

g=grammes ° C.=degrees centigrade

Unless otherwise stated, each concentration is expressed as percentage by weight.

### EXAMPLE 1

This Example illustrates the high solubility of each of a number of agrochemical active ingredients in Compound Number1 [DEET™].

A glass vial was approximately one eighth filled with an active ingredient [Al] and then solvent [in this example, Compound Number 1 from Table 1] was added until the vial was approximately one third full. The resultant sample was mixed with a Whirlimixer™ and was then stored at 25° C. The sample was checked every few days; if there was no solid active ingredient present then additional active ingredient was added; if there was no liquid remaining then additional solvent was added. This procedure was repeated until the sample had equilibrated for 4 weeks following the final addition of either active ingredient or solvent. The supernatent liquid layer was then analysed by gas chromatography [except for 4-hydroxy-3-[2-(2-methoxyethoxymethyl)-6-trifluoromethylpyridine-3-carbonyl]-bicyclo[3.2.1]oct-3-en-2-one which was analysed by liquid chromatography] for active ingredient concentration; the results are given in Table 2:

TABLE 2

| | Solubility in Compound |
|---|---|
| AI | No. 1 at 25° C. (% w/w) |
| Difenoconazole | 35.7 |
| Cyprodinil | 61.9 |
| 4-Hydroxy-3-[2-(2-methoxyethoxymethyl)-6- | 24.7 |
| trifluoromethylpyridine-3-carbonyl]- | |
| bicyclo[3.2.1]oct-3-en-2-one | |
| Azoxystrobin | 11.3 |
| Cyproconazole | 33.4 |

### EXAMPLE 2

This Example illustrates that Compound Number 1 is unusually effective at dissolving pesticides given that it has a water solubility which is low. Table 3 shows the aqueous solubility and pesticide solubility for several solvents. As can be seen the water soluble solvents, those with an aqueous solubility greater than 0.1% w/w in water, are more able to dissolve pesticides than the oils.

TABLE 3

| | Aqueous | A.I. Solubility at 25° C. (% w/w) | | |
|---|---|---|---|---|
| | solubility | Azoxy- | Cypro- | |
| Solvent | % w/w | strobin | conazole | Cyprodinil |
| N-Methyl pyrrolidone | 100 | 41.5 | 60.6 | 67.2 |
| DMSO | 100 | 32.1 | 55.5 | 69.0 |
| Tetrahydrofurfuryl alcohol | 100 | 11.1 | 39.8 | 55.7 |
| Solvesso ™ 200 ND/ULN | 0.1 | 5.6 | 10.4 | 34.4 |
| n-Butylbenzoate | 0.1 | 4.0 | 11.9 | 35.5 |
| Trimethylbenzene | 0.1 | 1.7 | 5.8 | 31.3 |
| Compound No. 1 | 0.1 | 11.3 | 33.4 | 61.9 |
| Fatty acid (C6-C10) methyl | 0.040 | 1.8 | 10.4 | 34.2 |
| ester (methyl caprylate) | | | | |
| Menthone | 0.030 | 3.2 | 19.1 | 42.3 |
| Exxate ™ 700 (heptyl | 0.030 | 1.7 | 11.4 | 34.5 |
| acetate) | | | | |
| Solvesso ™ 100 ND/ULN | 0.005 | 1.7 | 5.8 | 31.1 |
| Isobornyl acetate | 0.003 | 1.6 | 12.7 | 32.8 |
| Exxsol ™ D-80 (Dodecane) | 0.001 | 0.1 | 0.5 | 4.0 |

1. A composition comprising an agrochemical and a compound of formula (I) 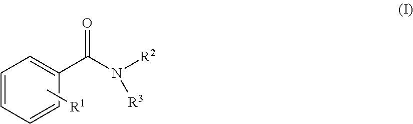
where R¹ is fluoro, methoxy or C₁₋₄ alkyl; R² and R³ are each independently optionally substituted C₁₋₆ alkyl or optionally substituted C₂₋₆ alkenyl; or R² is hydrogen and R³ is optionally substituted C₅₋₈ alkyl or optionally substituted C₄₋₆ alkenyl or optionally substituted benzyl; or R² and R³ and the nitrogen atom to which they are both attached together form an optionally substituted 5-, 6- or 7-membered ring, which ring optionally contains at least one further heteroatom selected from O, N and S.

2. A composition as claimed in claim 1 where the compound of formula (I) has an aqueous solubility at 25° C. which is less than 5% w/w.

3. A composition as claimed in claim 1 where the solubility of the agrochemical in the compound of formula (I) at 25° C. is greater than 5% w/w.

4. A composition as claimed in claim 1 where R¹ is in the ortho- or meta-position.

5. A composition as claimed in claim 1 where R¹ is fluoro, methoxy or C₁₋₂ alkyl.

6. A composition as claimed in claim 1 where R² and R³ together comprise one to four carbon atoms.

7. A compound of formula (I) as defined in claim 1 provided (i) that when R¹ is methyl in the meta-position, then R² is not ethyl when R³ is ethyl; and (ii) that when R¹ is methyl in the ortho-position, then R³ is not n-pentyl nor benzyl when R² is hydrogen; R³ is not n-propyl when R² is n-propyl; R² is not methyl when R³ is tert-butyl; R³ is not methyl when R² is tert-butyl; and R² and R³ and the nitrogen atom to which they are both attached do not together form pyrrolidinyl, morpholinyl or 2,6-dimethylmorpholinyl.

8. A process for preparing a compound of formula (I) as defined in claim 7 comprising the step of reacting a compound of formula (II) with a compound of formula (III) 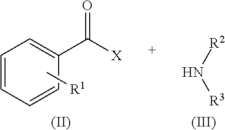
where X is Cl, OY or 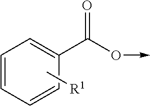
and Y is a leaving group; and R¹, R² and R³ are as defined in claim 7.

9-11. (canceled)

12. A method of forming an emulsifiable concentrate or an emulsion, the method comprising using a compound of formula (I) as a solvent.

13. The method of claim 12, wherein the emulsifiable concentrate or emulsion is an agrochemical formulation.
